(19) United States
(12) Patent Application Publication
(10) Pub. No.: US 2010/0082368 A1
(43) Pub. Date: Apr. 1, 2010

(54) WRONG SITE SURGERY PREVENTION SYSTEM

(75) Inventors: Gary R. Gecelter, Great Neck, NY (US); Jerome B. Korten, New York, NY (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: CORQUALITY SYSTEMS, INC., Great Neck, NY (US)

(21) Appl. No.: 12/240,030

(22) Filed: Sep. 29, 2008

(51) Int. Cl. A61B 5/00

(52) U.S. Cl. 705/3

(57) ABSTRACT

Compliance with pre-operative policies and procedures is ensured to prevent wrong site surgeries. Data related to a scheduled procedure is stored on a device. The data includes at least (1) indicia of personal identification for a patient and one or more participants in the scheduled procedure, and (2) information about the scheduled procedure. An input is received on the device that associates the patient with the data stored on the device. The patient and each of the one or more participants are then prompted to verify the indicia of personal identification and information about the scheduled procedure on the device. When the patient and one or more participants have all verified their respective indicia of personal identification and the information about the scheduled procedure, the device is operable to enable operating room functionality.

### CROSS-REFERENCE TO RELATED APPLICATION(S)

This application is related to co-pending application Ser. No. 11/825,896, entitled “Method and System for Ensuring Compliance with Mandated Pre-Operative Policies and Procedures to Prevent Wrong-Site Surgeries,” which is hereby incorporated by reference in its entirety.

### TECHNICAL FIELD

The present invention relates generally to hospital and operating room systems and methods. More particularly, the present invention relates to systems and methods related to assuring compliance with pre-operative policies and procedures to prevent wrong site surgeries.

### BACKGROUND

Wrong site surgery may include any of (a) the wrong operation performed on a patient, (b) an operation performed on the wrong patient, or (c) an operation performed on the wrong body part. Wrong site surgery is characterized as a preventable medical error, and thus great efforts are being made to reduce the incidence of wrong site surgery.

Numerous infamous cases of wrong site surgeries have caused much attention to be focused on the need to prevent or deter surgeries from being performed on the wrong limbs or body parts. Various factors are believed to contribute to the occurrence of wrong-site surgeries including failures of communication, documentation, and/or preoperative assessment; the involvement of more than one surgeon in a particular case; the need to perform multiple surgical procedures on a patient at one time, particularly on different sides of the patient's body; time pressures involving a hastening of start times and/or preoperative procedures; and unusual patient characteristics, such as physical deformity or obesity, which alter the usual processes for equipment set-up, patient preparation and/or patient positioning.

The Joint Commission on Accreditation of Healthcare Organizations, Sentinel Event Alert, issue six, Aug. 28, 1998, offers the following possible strategies for reducing the risk of wrong-site surgery: clearly mark the operative site and involve the patient in the marking process to enhance the reliability of the process; require an oral verification of the correct site in the operating room by each member of the surgical team; develop a verification checklist that includes all documents referencing the intended operative procedure and site, including the medical record, X-rays, and other imaging studies, and their direct observation of the marked operative site on the patient; personal involvement of the surgeon in obtaining informed consent; ensure through ongoing monitoring that verification procedures are followed for high-risk procedures; and “time out” immediately before starting the procedure.

Despite the implementation of strategies to prevent wrong site surgery, regrettably this seemingly most preventable of complications still occurs. Current methodologies to prevent wrong site surgery include the use of indelible markers and adhesive labels to identify appropriate surgical sites, a microchip attached to the patient at the anatomical location of the intended surgery which is scanned to retrieve electronically stored information prior to surgery to correlate a patient with the surgery to be performed, and patient safety alerting systems and methods including a display device for displaying a set of indicators, each indicator displaying one of a plurality of indicator states, each of the indicator states indicating a level of compliance with a patient safety procedure. These methodologies have inherent deficiencies and wrong site surgery continues to occur. A zero-tolerance policy for wrong site surgery has been mandated by major agencies and professional organizations that accredit health care providers. In accordance with this policy, the term “Never Event” has been coined by the National Quality Forum (www.qualityforum.org). Therefore, a need exists for better methods to further reduce the likelihood of wrong site surgery.

### SUMMARY

One aspect of the present invention relates to ensuring compliance with pre-operative policies and procedures is ensured to prevent wrong site surgeries. Data related to a scheduled procedure is stored on a device. The data includes at least (1) indicia of personal identification for a patient and one or more participants in the scheduled procedure, and (2) information about the scheduled procedure. An input is received on the device that associates the patient with the data stored on the device. The patient and each of the one or more participants are then prompted to verify the indicia of personal identification and information about the scheduled procedure on the device. When the patient and one or more participants have all verified their respective indicia of personal identification and the information about the scheduled procedure, the device is operable to enable operating room functionality.

In another aspect of the present invention, operating room functionality is controlled to ensure compliance with pre-operative policies and procedures designed to prevent wrong site surgeries. The operating room functionality is initialized to a disabled state. A home screen is displayed including a selectable patient interface module and a plurality of selectable participant interface modules. Each participant interface module is associated with a participant in a scheduled procedure to be performed on a patient. A patient verification screen is displayed when the patient interface module is selected. The patient verification screen includes indicia of patient personal identification and information about the scheduled procedure. One or more inputs are received from a patient to verify the indicia of patient personal identification and information about the scheduled procedure. A participant verification screen is displayed when a participant interface module is selected. The participant verification screen displayed is specific to the participant associated with the selected participant interface module and includes indicia of participant personal identification and information about the scheduled procedure. One or more inputs are received from the participant associated with the selected participant interface module to verify the indicia of participant personal identification and information about the scheduled procedure. A selectable activation module is displayed if the patient verifies the information on the patient verification screen and each participant verifies the information on the participant verification screen specific to the participant. When the activation module is selected, the activation module enables the operating room functionality.

In a further aspect of the present invention, a system controls operating room functionality to ensure compliance with pre-operative policies and procedures designed to prevent wrong site surgeries. The system includes a server operable to store data related to a plurality of scheduled procedures. For each scheduled procedure, the data includes at least (1) indicia of personal identification for a patient and a plurality of participants in the scheduled procedure, and (2) information about the scheduled procedure. The system also includes a remote device operable to receive data related to a scheduled procedure from the server and prompt each of the patient and plurality of participants to verify the indicia of personal identification and information about the scheduled procedure. The remote device facilitates enablement of the operating room functionality when the patient and plurality of participants have all verified their respective indicia of personal identification and the information about the scheduled procedure.

In a still further aspect of the present invention, a device controls operating room functionality to ensure compliance with pre-operative policies and procedures designed to prevent wrong site surgeries. The device includes a controller operable to receive and store data related to a scheduled procedure. The data related to the scheduled procedure includes at least (1) indicia of personal identification for a patient and one or more participants in the scheduled procedure, and (2) information about the scheduled procedure. The device also includes at least one input element operable to receive one or more inputs from each of the patient and plurality of participants to verify the indicia of personal identification and information about the scheduled procedure. The at least one input element is further operable to enable operating room functionality when the patient and plurality of participants have all verified their respective indicia of personal identification and the information about the scheduled procedure.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of an operating room including components controllable in accordance with the principles of the present invention.

FIG. 2 is a block diagram of a system configured to facilitate control of operating room components according to an embodiment of the present invention.

FIG. 3 is a block diagram of a remote device configured for use in the system of FIG. 2.

FIG. 4 is a flow diagram of a process for ensuring compliance with pre-operative policies and procedures to prevent wrong site surgeries according to an embodiment of the present invention.

FIGS. 5A-5I are illustrations of exemplary interfaces provided on a patient-associated remote device in the system of FIG. 3 during the process described in FIG. 4, according to an embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

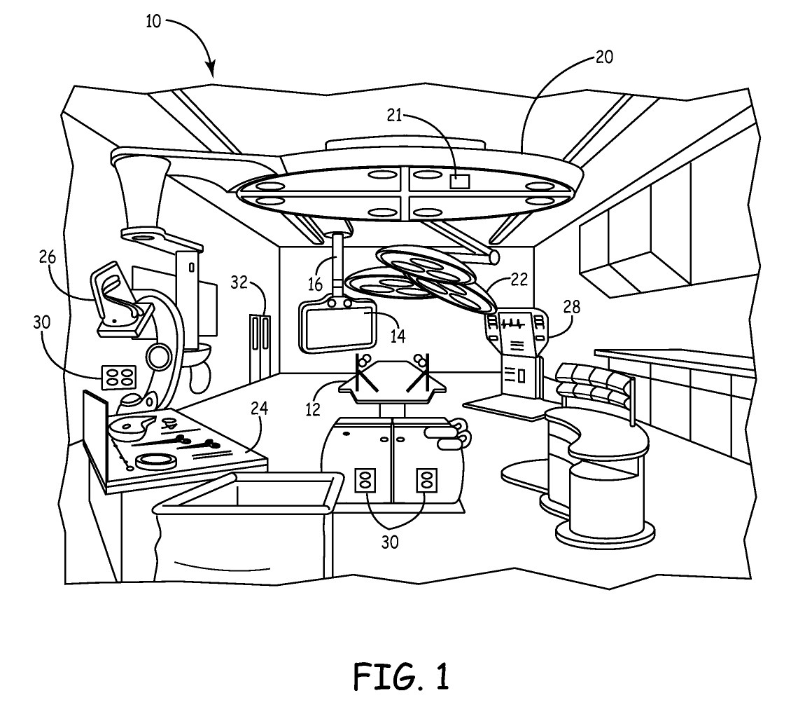

FIG. 1 is a perspective view of operating room 10 including components controllable in accordance with the principles of the present invention. Operating room 10 includes operating table 12, display 14 supported by boom 16, room light fixture 20, light sensor 21, surgical light fixture 22, instrument table 24, surgical microscope 26, and patient monitor 28. Display 14 is disposed proximate operating table 12 and may provide, for example, information related to a scheduled procedure or video from an instrument such as surgical microscope 26. Room light fixture 20 provides light throughout operating room 10, and surgical light fixture 22 provides high-intensity light in the area surrounding operating table 12. Room light sensor 21 is operable to generate and transmit a signal in response to sensing light, which indicates that room light fixture 20 and/or surgical light fixture 22 have been turned on.

Operating room 10 also includes a plurality of electrical outlets 30 that are suitable for providing electrical power to surgical instruments and other electrical components in operating room 10. For example, surgical microscope 26 and patient monitor 28 may be powered through electrical outlets 30. The configuration, location, and number of electrical outlets 30 shown in FIG. 1 are merely illustrative, and any arrangement and type of electrical outlets may alternatively be provided in operating room 10. In alternative embodiments, surgical microscope 26 and patient monitor 28 are hard wired to the electrical supply in operating room 10.

Doors 32 provide access to operating room 10. In some embodiments, doors 32 include an electrical and/or mechanical locking mechanism that may be controlled locally or remotely to limit access to operating room 10.

As will be described in more detail herein, the present invention relates to controlling functionality of operating room 10 to assure compliance with pre-operative policies and procedures. The pre-operative policies and procedures are designed to prevent wrong site surgery, such as the wrong operation performed on a patient, an operation performed on the wrong patient, and/or an operation performed on the wrong body part of a patient. For example, controlling the functionality of operating room 10 may include controlling operation of electrical components in operating room 10, such as display 14, room light fixture 20, surgical light fixture 22, surgical microscope 26, and/or patient monitor 28. That is, the electrical components in operating room 10 may be disabled until the proper pre-operative steps have been taken. As another example, controlling functionality of operating room 10 may include controlling operation of electrical outlets 30 such that electricity is not supplied to equipment plugged into electrical outlets 30 until compliance with the pre-operative policies and procedures. As a further example, controlling functionality of operating room 10 may include controlling doors 32 such that doors 32 are locked and access to operating room 10 is prevented until certain pre-operative steps are completed. The components shown in operating room 10 are merely exemplary, and the present invention contemplates control of any portion of operating room 10 or the equipment therein to prevent a procedure from being performed without compliance with the pre-operative policies and procedures. In addition, it should be noted that the term “operating room” as used herein encompasses any environment in which surgery or other operative procedures are performed on a patient to investigate and/or treat a pathological condition.

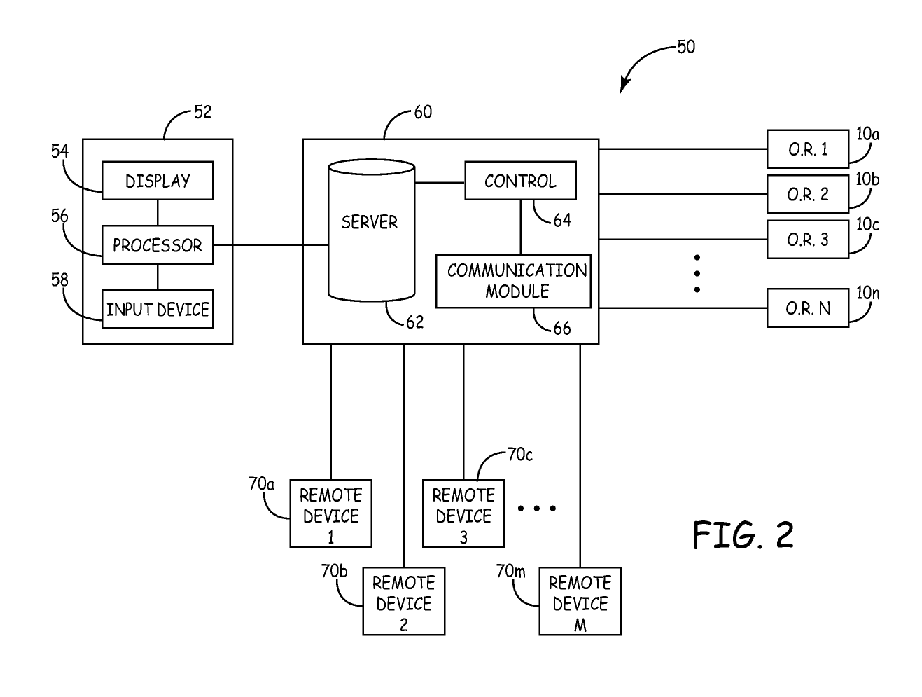

FIG. 2 is a block diagram of system 50 operable to ensure compliance with mandated pre-operative policies and procedures to prevent wrong site surgeries, according to an embodiment of the present invention. System 50 includes data entry computer 52 including display 54, processor 56, and input device 58. Processor 56 communicates with display 54 and input device 58 in data entry computer 52. System 50 also includes system control 60 including server 62, central controller 64, and communication module 66. Server 62 communicates with processor 56 and controller 64, and controller 64 communicates with communication module 66. System control 60 is configured to communicate with a plurality of remote devices 70a, 70b, 70c, . . . , 70m. System control 60 is also configured to communicate with and control components of operating rooms 10a, 10b, 10c, . . . , 10n, such as those discussed above with regard to FIG. 1. It should be noted that any number of remote devices 70 and operating rooms 10 may be associated with system 50, and the use of the letter “m” in association with remote device 70m and the letter “n” in association with operating room 10n is only for illustration and is not indicative of the number of each of these elements of system 50.

Data entry computer 52 is operable to provide information to and receive information from system control 60, and in particular server 62. Processor 56 is configured to receive information input through input device 58 and, in some cases, present the input information on display 54. In some embodiments, computer 52 is a personal computer, wherein display 54 comprises a monitor, processor 56 comprises a central processing unit, and input device 58 comprises a keyboard. Input device 58 may also comprise other types of input devices, such as a biometric input device, a personal digital assistant, a disk drive, and so on.

Data entry computer 52 may be used, for example, to enter information relating to scheduled surgical procedures for storage in server 62. This information, which may be entered by a scheduling nurse based on a scheduling order from a surgeon, may include identification information for the patient (e.g., name, date of birth, etc.), the type of surgical procedure to be performed, the location of the procedure on the patient's body, the time and date of the procedure, and personnel involved with the scheduled procedure. Data entry computer 52 may also be used to enter and manage lists of operating room personnel who are registered to participate in procedures performed in operating rooms 10a-10n. Furthermore, data entry computer 52 may be used to receive indicia of personal identification from patients and operating room personnel for storage in server 62, including biometric information such as fingerprint scan data, iris scan data, voice recognition data, DNA data, and the like. Data entry computer 52 may communicate with server 60 via either wired or wireless communication.

Server 62 is configured to store information in addition to the information received from data entry computer 52. For example, server 62 may store software or other programming code executable by controller 64 during operation of system 50. In some embodiments, server 62 stores executable software modules to transmit information to remote devices 70a-70m related to scheduled surgical procedures, compare indicia of personal identification received from any of remote devices 70a-70m with indicia of personal identification stored in server 62, and/or manage operation of components in operating rooms 10a-10n.

Controller 64, which may be a microprocessor based device, controls operation of system 50 by coordinating information communicated between system control 60, operating rooms 10a-10n, and remote devices 70a-70m, and by processing this information to control components of operating rooms 10a-10n to assure compliance with pre-operative policies and procedures. In particular, controller 64 executes the software or other programming code stored on server 62 discussed above, and communicates with operating rooms 10a-10n and remote devices 70a-70m via communication module 66.

Communication module 66 may be any type of interface operable to communicate information to and from system control 60. In some embodiments, communication module 66 includes a wireless transducer operable to transmit and receive signals over a short or long range wireless communication protocol, such as WiFi, radio frequency (RF), Bluetooth, or the like. In other embodiments, communication module 66 is a network interface connected via a wired connection to other components in system 50.

Remote devices 70a-70m are electronic devices that may be configured to communicate with system control 60 wirelessly or via a wired network. Each remote device 70a-70m provides an interface between system control 60 and the patient and personnel associated with a scheduled procedure. To this end, remote devices 70a-70m are each operable to verify at least indicia of personal identification for the patient and the participants in the scheduled procedure (e.g., surgeon, anesthesiologist, nurse, etc.) and information about the scheduled procedure. Remote devices 70a-70m may obtain this information related to a scheduled procedure from server 62 for storage locally on the remote device 70. In some embodiments, each remote device 70a-70m is associated with a patient when or shortly after the patient arrives at a medical facility, and the remote device 70 associated with the patient remains with the patient for the patient's entire visit to the medical facility. For example, a patient may provide some indicia of personal identification to remote device 70, which will cause remote device 70 to obtain information about that patient's scheduled procedure from server 62. Alternatively, the information about the patient's scheduled procedure may be stored on remote device 70 prior to the patient's arrival to the medical facility, and the patient's indicia of personal identification may be used to associate the remote device 70 with that patient upon or after arrival to the medical facility. In either case, this assures that the information related to the scheduled procedure on each remote device 70a-70m remains with the correct patient.

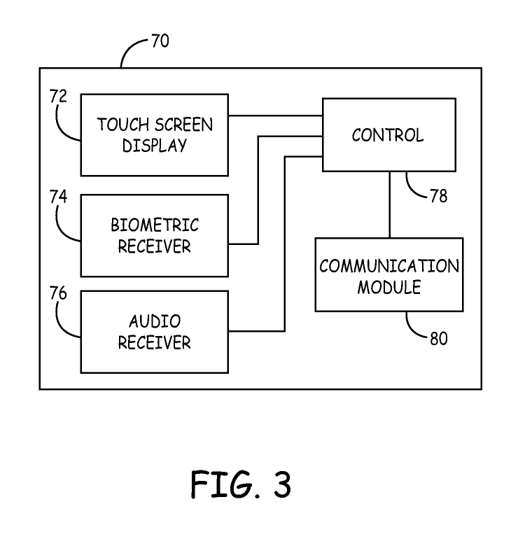

FIG. 3 is a block diagram of remote device 70 according to an embodiment of the present invention. Remote device 70 includes touch screen display 72, biometric receiver 74, audio receiver 76, controller 78, and communication module 80. Touch screen display 72, biometric receiver 74, and audio receiver 76, and communication module 80 each communicate with controller 78. In some embodiments, touch screen display 72, biometric receiver 74, audio receiver 76, controller 78, and communication module 80 are arranged in the form of a tablet personal computer or other portable processing device.

Touch screen display 72 provides an interface through which a patient and participants associated with the patient's scheduled procedure receive and respond to pre-operative instructions and procedures. For example, the patient and participants in the scheduled procedure may receive instructions to verify their respective identities to assure that the proper people are involved with the procedure. As another example, touch screen display 72 may provide an interface through which patients or participants verify information about the scheduled procedure, such as the surgery site. The interface may be an on-screen display that the patient and/or personnel interact with by touching appropriate portions of the display. In alternative embodiments, a display without touch screen capabilities is employed in remote device 70, with another device, such as a keyboard, providing the interface through which the patient and participants respond to on-screen instructions.

Biometric receiver 74 provides an interface through which the patient and participants submit a biometric input as an indication of personal identification. Biometric receiver 74 may be configured to receive any type of biometric information from the patient and personnel. Some representative types of biometric information that biometric receiver 74 may be configured to receive include, but are not limited to, fingerprint information, hand structure information, face structure information, retinal information, iris information, behavioral trait information, and/or signature pattern information.

Audio receiver 76 is a microphone or other acoustic device capable of receiving audio information for processing by controller 78. For example, audio receiver 76 may be used to record the voice of the patient or participants for identification purposes via voice recognition. Audio receiver 76 may also be employed during the pre-operative review of information related to the scheduled procedure as a further element of verification. For example, at the end of the pre-operative verification process, one or more participants in the scheduled procedure may be instructed to read information about the scheduled procedure and verify the information by audibly asserting the accuracy of the information.

Controller 78, which may include a microprocessor based device, controls operation of remote device 70 by coordinating information communicated between system control 60 and remote device 70, and by processing this information to interface with the patient and participants associated with the schedule procedure to assure compliance with pre-operative policies and procedures. Controller 78 may also include a memory component for storing data and information received from system control 60.

Communication module 80 may be any type of interface operable to communicate information with system control 60. In some embodiments, communication module 80 includes a wireless transducer operable to transmit and receive signals over a short or long range wireless communication protocol. The communication protocol used by communication module 80 typically matches the communication protocol used by communication module 60. In other embodiments, communication module 80 is a network interface connected via a wired connection to system control 60.

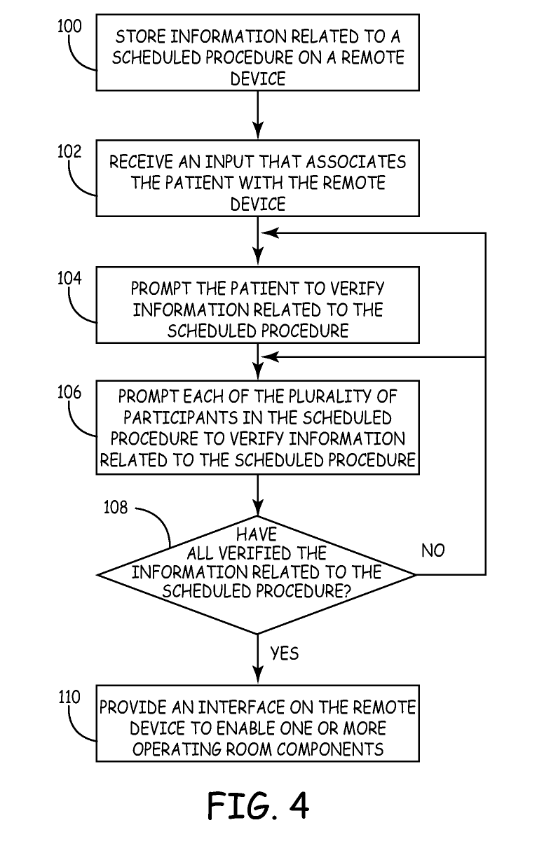

FIG. 4 is a flow diagram of a process for ensuring compliance with pre-operative policies and procedures to prevent wrong site surgeries, according to an embodiment of the present invention. Prior to commencement of the process illustrated in FIG. 4, the functionality or functional access of operating room 10 assigned for a scheduled procedure (e.g., one of operating rooms 10a-10n) may be disabled by controller 64. For example, operation of the electrical components in the assigned operating room 10 may be disabled, and/or doors 32 of the assigned operating room 10 may be locked prior to commencement of the pre-operative verification process. In some embodiments, the components that are initiated to the disabled state in operating room 10 are critical to performance of the scheduled procedure.

In step 100, information related to a scheduled procedure is stored on a remote device 70 (e.g., one of remote devices 70a-70m of system 50 shown in FIG. 2). The information may be retrieved from server 62 of system control 60 prior to or upon arrival of the patient to the medical facility. For example, system control 60 may be operated to send information related to the patient's scheduled procedure in response to commands from data entry computer 52.

The information related to the scheduled procedure may include indicia of personal identification for the patient and participants (i.e., personnel) in the scheduled procedure. The indicia of personal identification may include, for example, name, address, biometric information, and/or other identifying information for the patient and participants.

The information related to the schedule procedure may also include information about the scheduled procedure. For example, information about the scheduled procedure may include general and specific information about the anatomical site of the procedure, the type of procedure, the positioning of the anatomical site during the procedure, the positioning of the patient during the procedure, medical practices to be performed during the procedure (e.g., x-ray), the date and time of the procedure, the location of the procedure (e.g., operating room location in the medical facility), and medications, therapeutic substances, and/or anesthetic substances to be administered during the procedure. The information about the scheduled procedure may also include medical history and allergy information for the patient, and the role each of the participants have in the scheduled procedure (e.g., surgeon, nurse, anesthesiologist, etc.). This list is non-exclusive, and any type of information about the scheduled procedure may be included in the information stored in remote device 70.

In step 102, the patient in the scheduled procedure provides an input that associates the patient with remote device 70. In some embodiments, patient provides a biometric input via biometric receiver 74 of remote device 70 to associate the patient with remote device 70. When the patient is associated with remote device 70, remote device 70 remains with the patient for the patient's visit to the medical facility, or at least until commencement of the procedure. For example, remote device 70 may be attached to the patient's hospital bed or gurney, or the patient may carry remote device 70 on his or her person until the scheduled procedure starts. The goal is to assure that the remote device 70 carrying the information about the patient's scheduled procedure is not mistakenly placed with or given to a different patient.

In an alternative embodiment, steps 100 and 102 are reversed such that the patient is associated with remote device 70 first, and the information related to the patient's scheduled procedure is stored on remote device 70 after the patient is associated with the device. That is, a patient may be asked to provide an input (e.g., a biometric input) on one of remote devices 70a-70m upon arrival to the medical facility. Remote device 70 may then query controller 64 to find a match to the patient's input in server 62 (e.g., by running matching software stored on server 62). After controller 64 finds a match, information related to the scheduled procedure, which is associated with the patient's input and stored in server 62, is downloaded or otherwise obtained by remote device 70 from server 62.

In step 104, the patient is prompted to verify information related to the scheduled procedure. For example, the patient may be prompted on touch screen display 72 of remote device 70 to verify information related to the scheduled procedure by touching portions of the display. As another example, the patient may provide an audible response on audio receiver 76 that asserts the accuracy of the information in response to an audio prompt or a prompt on touch screen display 72. Step 104 may occur anytime between when the patient is associated with remote device 70 and when the patient is anesthetized for the scheduled procedure.

In some embodiments, information related to the scheduled procedure that the patient is prompted to verify includes indicia of the patient's personal identification and information about the scheduled procedure. For example, the patient may be prompted to again provide a biometric input via biometric receiver 74, especially if a relatively long period of time has passed between associating the patient with remote device 70 and commencement of the pre-operative verification process. The patient may also be prompted to verify other indicia of personal identification including, for example, the patient's name, date of birth, social security number, patient identification number, allergies, and/or other personal medical information. The information about the scheduled procedure that the patient is prompted to verify may include, for example, the anatomical site of the procedure, the type of procedure, the date and time of the procedure, and/or the name of the doctor performing the procedure.

In step 106, each of the participants in the scheduled procedure is prompted to verify information related to the scheduled procedure. In some embodiments, step 106 occurs a short time or immediately before the scheduled procedure. Each of the participants may be prompted on touch screen display 72 of remote device 70 to verify information related to the scheduled procedure by touching portions of the display. As another example, each of the participants may provide an audible response on audio receiver 76 that asserts the accuracy of the information in response to an audio prompt or a prompt on touch screen display 72. Each participant may be prompted to verify a set of information specific to the participant.

In some embodiments, information related to the scheduled procedure that each participant is prompted to verify includes indicia of the participant's personal identification and information about the scheduled procedure. For example, each participant may be prompted to provide a biometric input via biometric receiver 74. Each participant may also be prompted to verify other indicia of personal identification including, for example, the participant's name and employee identification number.

The information about the scheduled procedure that each participant is prompted to verify may include, for example, general and specific information about the anatomical site of the procedure, the type of procedure, the positioning of the anatomical site during the procedure, the date and time of the procedure, the patient's name and medical history information, and/or the names of other participants in the procedure. In addition, the information verified by each participant may be specific to the role of the participant in the scheduled procedure. For example, the surgeon performing the scheduled procedure may be prompted to verify more information specific to the procedure itself, while the anesthesiologist may be prompted to verify more information about the anesthetics to be used during the procedure.

In decision step 108, remote device 70 determines whether the patient and all participants in the scheduled procedure have verified the information related to the scheduled procedure. If any of the information related to the scheduled procedure has not been verified by the patient, the process returns to step 104 to prompt the patient to verify any unverified information. If any of the information related to the scheduled procedure has not been verified by any of the participants, the process returns to step 106 to prompt those participants to verify any unverified information. In some embodiments, if any of the information related to the scheduled procedure is changed or not confirmed by the patient or any of the participants, the process returns to steps 104 and/or 106 to have each of the patient and participants re-verify the changed or unconfirmed information.

If remote device 70 determines that the patient and all participants in the scheduled procedure have verified the information related to the scheduled procedure, then, in step 110, remote device 70 provides an interface to enable one or more operating components in operating room 10. For example, the interface may be in the form of a button graphic on touch screen display 72 that is selectable or actuatable by touching portions of the display within the button graphic. When the interface is actuated on remote device 70, a signal is sent to system control 60 that causes controller 64 to enable the functionality or functional access of operating room 10 that is assigned for the scheduled procedure (e.g., one of operating rooms 10a-10n). For example, actuating the interface on touch screen display 72 may enable any of display 14, room light fixture 20, surgical light fixture 22, surgical microscope 26, and patient monitor 28, and/or electrical outlets 30 in operating room 10 (FIG. 1). As another example, actuating the interface on touch screen display 72 may unlock doors 32 to provide access to operating room 10. After the functionality of operating room 10 has been enabled, the scheduled procedure can commence. In an alternative embodiment, one or more of the participants are prompted to perform a final pre-operative verification after actuating the interface on touch screen display 72 and prior to enabling the functionality of operating room 10.

It should be understood that variations on and additions to this process are contemplated by the present invention. For example, safeguards may be integrated into system 50 to assure that the pre-operative process described above is not bypassed. In some embodiments, system 50 is configured such that room light sensor 21 as shown in FIG. 1 is arranged to sense light from light fixtures 20 and/or 22, and operable to generate and transmit a signal to system control 60 or remote device 70 in response to sensing light. If a signal is received from room light sensor 21 prior to completing the process described above, a notification may be sent alerting the participants or others in the medical facility that the lights were turned on prior to completing the process. An audible alarm may also sound to deter the participants from trying to proceed with the procedure without completing the pre-operative process.

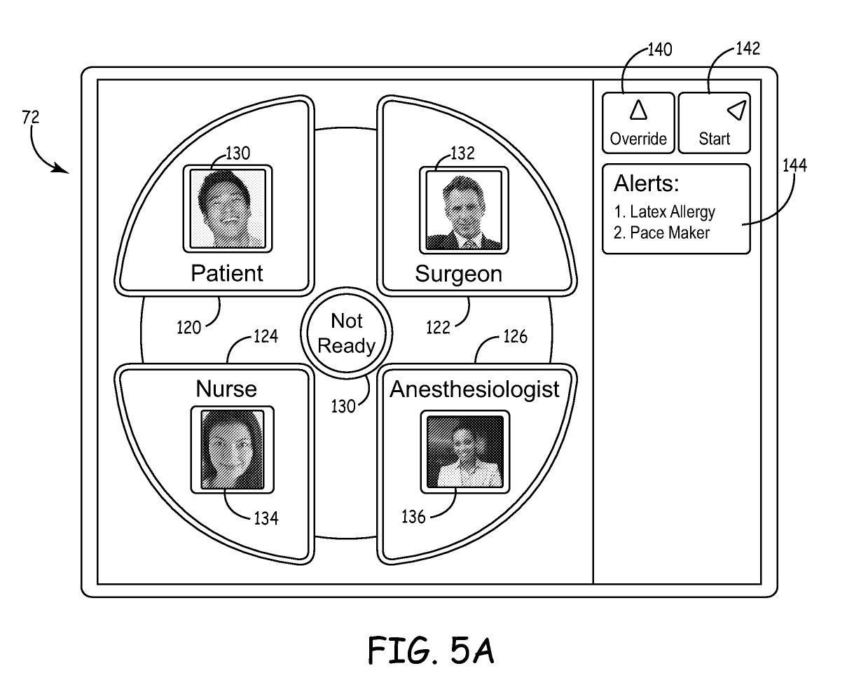

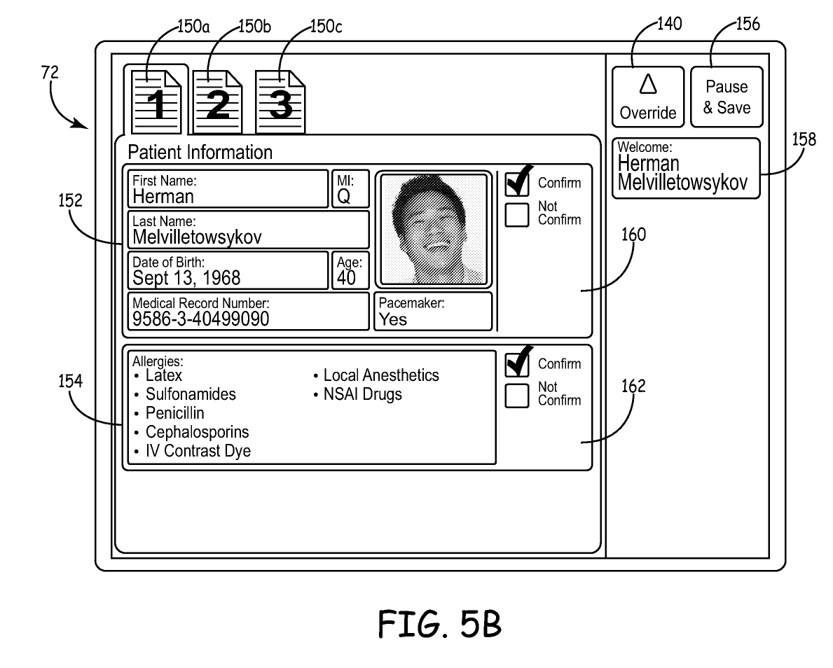

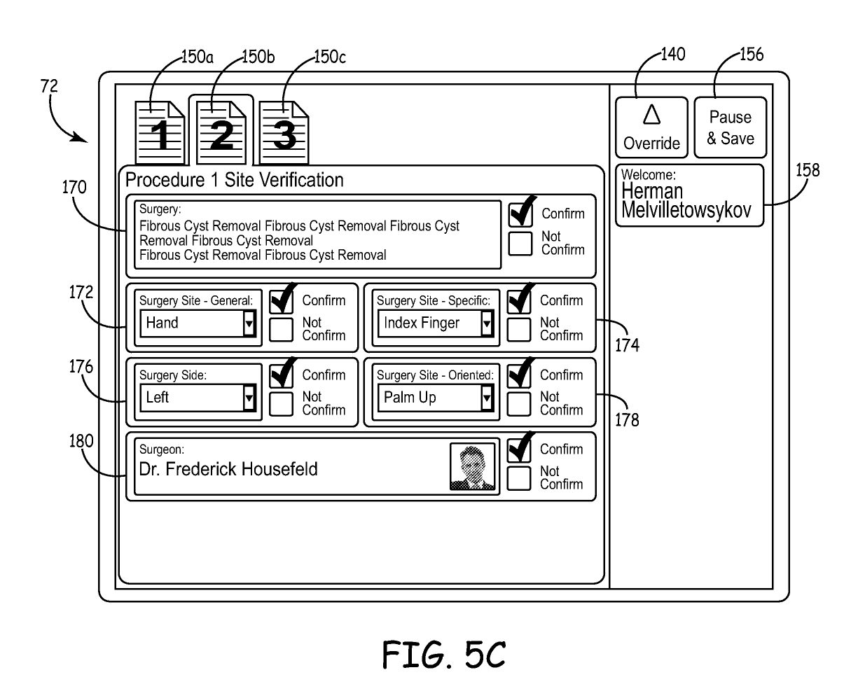

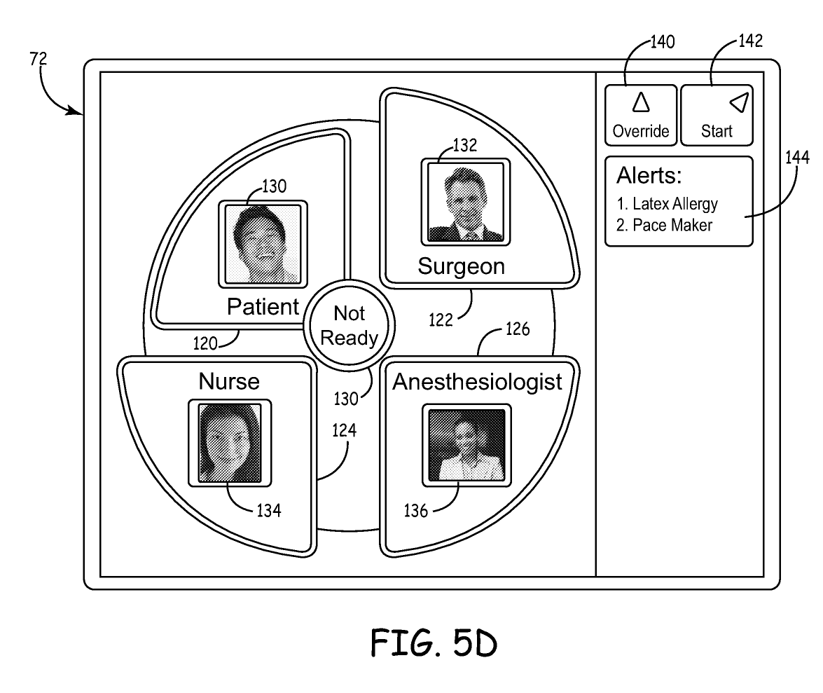

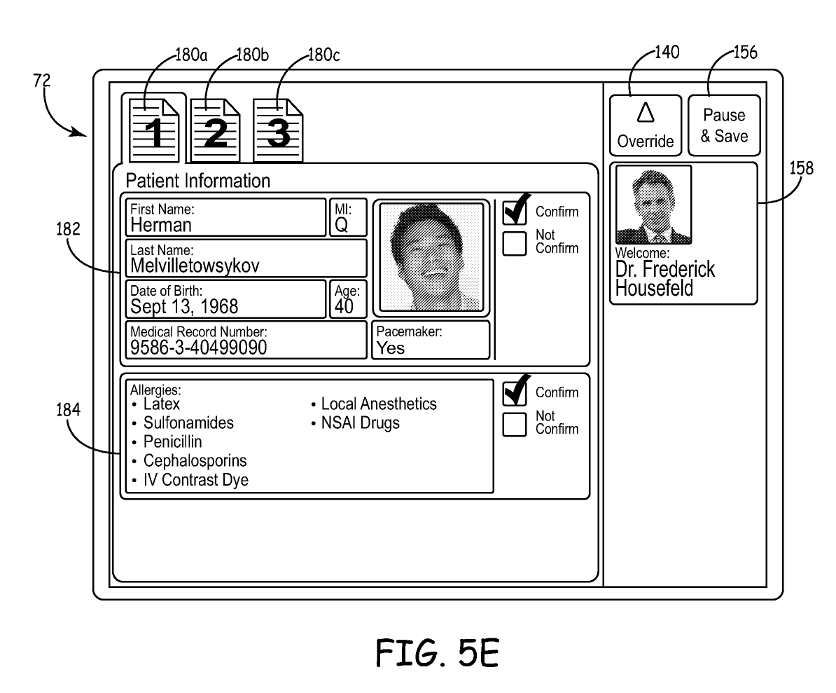

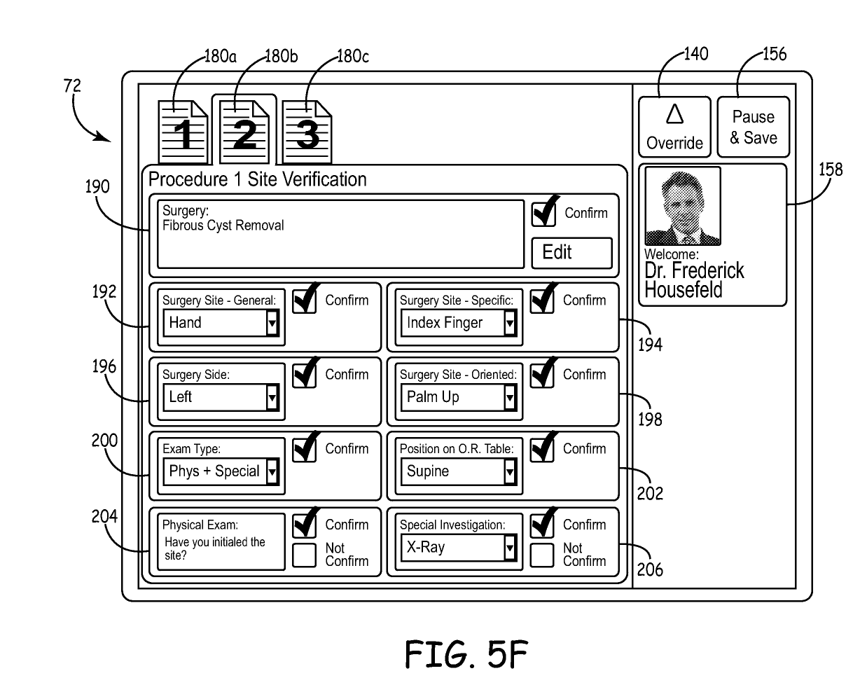

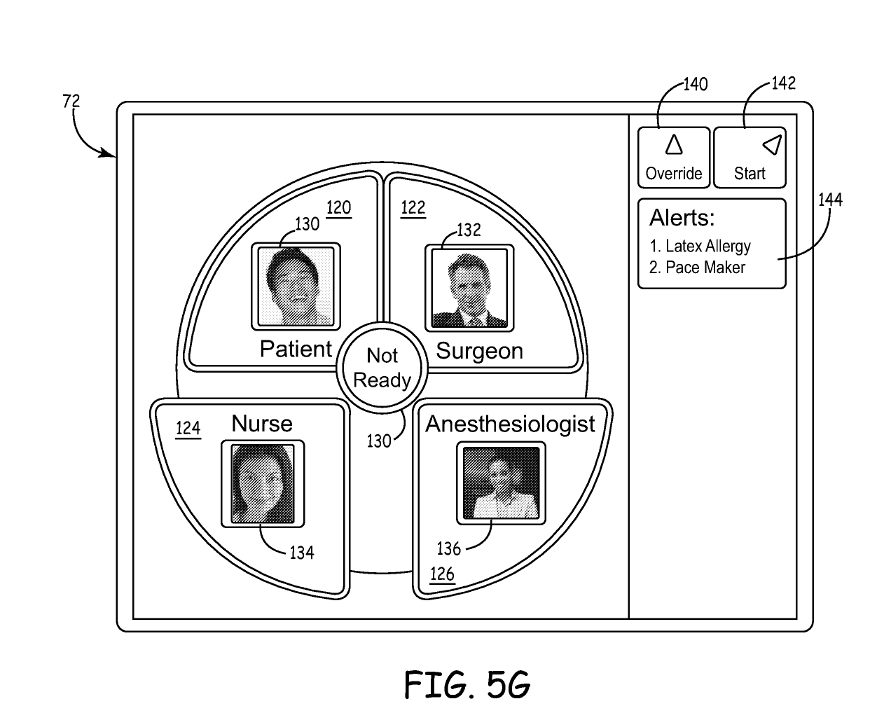

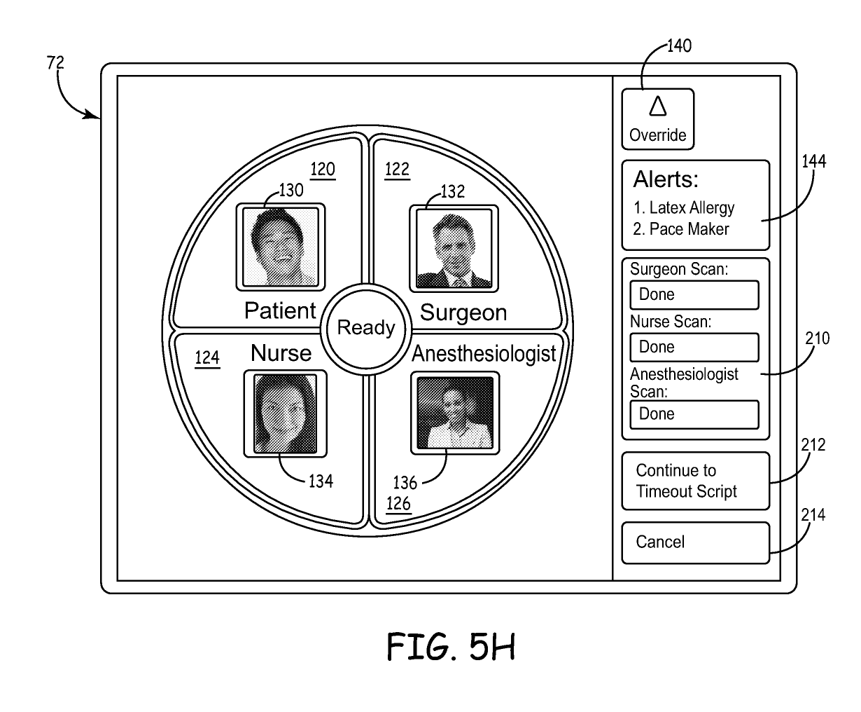

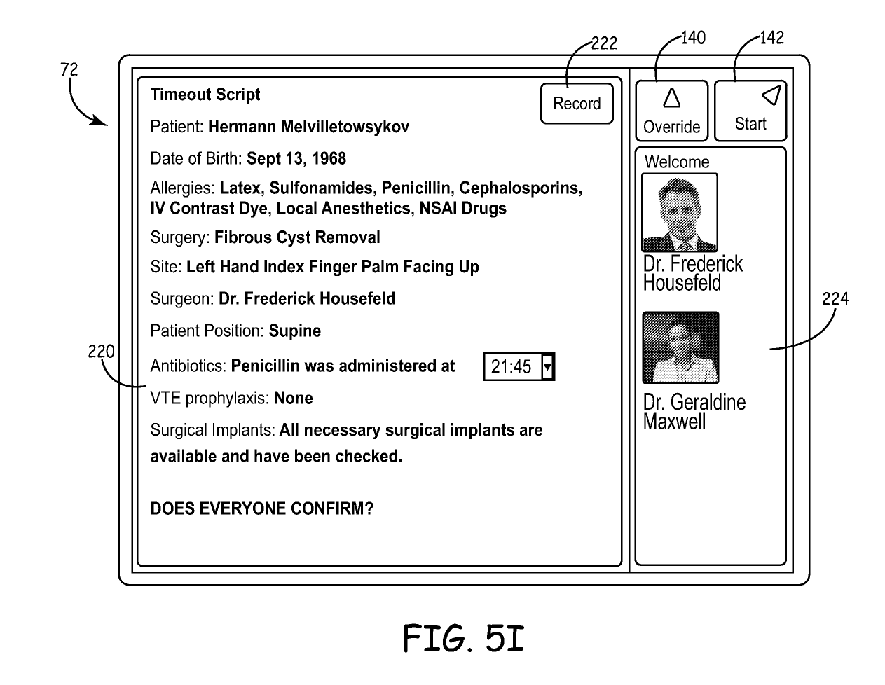

To illustrate how the process of prompting the patient and participants in the scheduled procedure may appear on touch screen display 72, FIGS. 5A-5I are exemplary screen shots of touch screen display 72 that may appear during the process described in FIG. 4. FIG. 5A is a screen shot of a home screen on touch screen display 72 that may be the first screen the patient and plurality of participants see after the patient is associated with remote device 70. The home screen includes patient module 120, surgeon module 122, nurse module 124, and anesthesiologist module 126. The modules shown are merely exemplary, and any number of modules associated with any participants in the scheduled procedure may be included on the home screen. In essence, each person involved with the scheduled procedure who needs to verify information related to the scheduled procedure may have a module on the home screen. In some embodiments, each category of people involved in the procedure (e.g., patient, surgeon, nurse, anesthesiologist) has a module, such that more than one person is associated with some of the modules. For example, if surgery on more than one anatomical part is being performed during a procedure, a surgeon for each procedure may be associated with the “Surgeon” module. In any case, each module 120, 122, 124, and 126 may include one or more photographs 130, 132, 134, and 136, respectively, to identify the person or people associated with that module.

The home screen also includes operating room activation interface 130. At the outset of the process, activation interface 130 may display “Not Ready” indicating that the pre-operative process has not been completed. Activation interface 130 may also be initiated to a state such that, when pressed on touch screen display 72, nothing happens or a message appears indicating that the pre-operative process has not been completed. In order to indicate that the patient and/or participants in the scheduled procedure have not verified the information related to the scheduled procedure, modules 120, 122, 124, and 126, and activation interface 130 may be displayed in red, and/or modules 120, 122, 124, and 126 may be spaced from activation interface 130.

The home screen may further include override button 140, start button 142, and alert window 144. Override button 140, which may appear on all screens during the process, allows a participant in the scheduled procedure to interrupt the pre-operative verification process at any time, such as in the case of an emergency, to enable the disabled components in operating room 10. If override button 140 is pressed during the verification process, a signal may be sent to controller 64 to note that the verification process was bypassed. Start button 142 allows the patient or any of the participants to start the pre-operative verification process. Alert window 144 provides a field on the home screen that allows the surgeon or other medical professionals to view vital information to the scheduled procedure.

FIG. 5B is a screen shot of a patient personal identification screen that may be displayed on touch screen display 72 when the patient presses patient module 120 on the home screen shown in FIG. 5A. The patient personal information screen includes content tabs 150a, 150b, and 150c, personal identification module 152, and medical information module 154. The patient personal information screen is provided under content tab 150a of patient module 120. Also shown on the right side of the patient personal information screen are override button 140, pause and save button 156, and active reviewer box 158. Pause and save button 156 allows the active reviewer, shown in active reviewer box 158, to pause the verification process and save the progress made in the verification process.

In the embodiment shown, personal identification module 152 includes the patient's name, date of birth, age, medical record number, and whether the patient has a pacemaker implanted in his body. Medical information module 154 includes a list of pharmaceuticals and materials to which the patient is allergic. Personal identification module 152 also includes confirmation section 160, and medical information module 154 also includes confirmation section 162, each of which allows the patient to confirm or not confirm the information contained in each of the modules. For example, the patient may press touch screen display 72 in the box next to “Confirm” in confirmation section 160 to confirm the information in personal identification module 152. A check mark or other indicia may appear in the box next to confirm to show that the patient confirmed this information. The patient may be able to confirm or not confirm the information in both personal identification module 152 and medical information module 154 when on the patient personal identification screen, or the patient may need to confirm or not confirm the information in one of personal identification module 152 or medical information module 154 before being able to confirm in the other module. In some embodiments, personal identification module 152 and medical information module 154 change to a first color (e.g., green) when the information is confirmed, and to a second color (e.g., yellow) when the information is not confirmed. In addition, tab 150a may change to a first color (e.g., green) when all information is confirmed under that tab, and to a second color (e.g., yellow) when any of the information under that tab is not confirmed. These color based indicators on the screens under modules 120, 122, 124, and 126 are also applicable to any of the screens and modules described below or illustrated in FIG. 5A. The patient may also be prompted to provide a biometric input via biometric receiver 74 before, during, or after the verification process on the personal identification screen.

FIG. 5C is a screen shot of a patient procedure information screen that may be displayed on touch screen display 72 when the patient confirms or does not confirm the information on the patient personal identification screen shown in FIG. 5B. The patient procedure information screen is provided under content tab 150b of patient module 120. In the embodiment shown, the patient procedure information screen includes surgery description module 170, general surgery site description module 172, specific surgery site description module 174, surgery location module 176, surgery location orientation module 178, and surgeon module 180. Each of the modules includes a confirmation section similar to that associated with the modules on the patient personal identification screen. Thus, as on the patient personal identification screen, the patient may confirm or not confirm the information in each of the modules displayed.

FIG. 5D is a screen shot of the home screen that may be displayed after the patient has confirmed all information related to the scheduled procedure. As is shown, patient module 120 has moved toward the center of the home screen such that activation interface 130 overlaps patient module 120. In addition, patient module 120 may be displayed in a color different than modules 122, 124, and 126 (e.g., green) to indicate that the patient has confirmed all information during the verification process. If the patient has not confirmed any of the information during the verification process, patient module 120 may remain spaced from activation interface 130 (as shown in FIG. 5A) and displayed in another color (e.g., yellow), which indicates that one or more participants in the procedure need to confirm or not confirm the patient's unconfirmed information.

FIG. 5E is a screen shot of a patient personal identification screen that may be displayed on touch screen display 72 when the surgeon presses surgeon module 122 on the home screen. The patient personal information screen includes content tabs 180a, 180b, and 180c, patient identification module 182, and medical information module 184. The patient personal information screen is provided under content tab 180a of surgeon module 122. Also shown on the right side of the patient personal information screen are pause and save button 156 and active reviewer box 158. As discussed above, pause and save button 156 allows the active reviewer, shown in active reviewer box 158, to pause the verification process and save the progress made in the verification process. Similar to the patient personal identification screen discussed above with regard to FIG. 5B, the surgeon may confirm or not confirm the information contained in patient identification module 182 and medical information module 184.

FIG. 5F is a screen shot of a surgeon procedure information screen that may be displayed on touch screen display 72 when the surgeon confirms or does not confirm the information on the patient personal identification screen shown in FIG. 5E. The surgeon procedure information screen is provided under content tab 180b of surgeon module 122. The information on the surgeon procedure information screen is specific information that is to be confirmed or not confirmed by the surgeon. For example, the information provided on the surgeon procedure information screen for confirmation is more detailed with regard to the surgery than other participants in the procedure, while the corresponding screen in anesthesiologist module 126 would have less detail about the surgery and more detail about the anesthesia involved in the procedure.

In the embodiment shown, the surgeon procedure information screen includes surgery description module 190, general surgery site description module 192, specific surgery site description module 194, surgery location module 196, surgery location orientation module 198, examination type module 200, patient position module 202, physical examination verification module 204, and special investigation module 206. The surgeon may review the information provided in each of the modules and confirm its accuracy or correct the information in the module and subsequently confirm the changed information. If the surgeon changes the information in any of the modules, the patient and/or other participants in the procedure may be prompted to re-confirm the accuracy of the changed information.

The modules on the surgeon procedure information screen may be presented one at a time for review, confirmation, and, if necessary, updating by the surgeon so that each module is reviewed and considered before moving on to the next module. In some embodiments, the order the modules are presented for review to the surgeon changes between procedures so that the surgeon is forced to review and consider the information in the module prior to confirming the information. This same approach may be applied to the procedure information verification presented to the other participants in the procedure. This “forcing function” prevents the parties involved in the procedure from starting the procedure without consciously considering each element of the pre-operative verification process.

FIG. 5G is a screen shot of the home screen that may be displayed after the patient and surgeon have confirmed all information related to the scheduled procedure. As is shown, patient module 120 and surgeon module 122 has moved toward the center of the home screen such that activation interface 130 overlaps patient module 120 and surgeon module 122. In addition, patient module 120 and surgeon module 122 may be displayed in a color different than modules 124 and 126 (e.g., green). However, because the nurse and anesthesiologist have not confirmed the information in their respective modules 124 and 126, activation interface still displays “Not Ready.”

If the patient or surgeon has not confirmed any of the information during the verification process, or if the patient or surgeon need to re-confirm any information, patient module 120 or surgeon module 122 may remain spaced from activation interface 130 and displayed in another color (e.g., yellow), which indicates that a participant in the procedure needs to confirm or not confirm the information.

The verification process for the nurse and anesthesiologist that is displayed when nurse module 124 and anesthesiologist module 126, respectively, are pressed may be similar to that described with regard to surgeon module 122, wherein the patient personal identification information and information about the procedure are verified. As discussed above, the information about the scheduled procedure that is verified by each participant may differ based on the role the participant has in the scheduled procedure.

FIG. 5H is a screen shot of the home screen that may be displayed when the patient and all participants have confirmed all information related to the scheduled procedure. As is shown, patient module 120, surgeon module 122, nurse module 124, and anesthesiologist module 126 have all moved toward the center of the home screen such that activation interface 130 overlaps modules 120, 122, 124, and 126. In addition, all modules 120, 122, 124, and 126 may be displayed in the same color (e.g., green). Activation interface 130 also displays “Ready” and may be displayed in the same color as modules 120, 122, 124, and 126 (e.g., green). When activation interface 130 is pressed on touch screen display 72, a signal is sent to system control 60 to enable the one or more components in operating room 10 that were initiated to a disabled state.

Also shown in the screen shot of FIG. 5H are scan verification window 210, timeout script activation button 212, and cancel button 214. Scan verification window 210 indicates whether each of the participants to the scheduled procedure have provided an indicia of personal identification to verify his or her identity, such as a biometric input on biometric receiver 74. If scan verification window 210 indicates that one or more of the participants have not provided an indicia of personal identification, enablement of the one or more components in operating room 10 may be prevented by system control 60 or remote device 70.

In some embodiments, the participants in the scheduled procedure may be prompted to review the timeout script by pressing timeout script activation button 212. The timeout script is a summary of information relating to the scheduled procedure, and may be used as a final verification of the information prior to commencement of the procedure. Cancel button 214 may be pressed to skip the review of the timeout script if this step is not required as part of the pre-operative verification process.

FIG. 5I is a screen shot of the timeout script screen that may be displayed if timeout script activation button 212 is pressed in FIG. 5H. The timeout script screen includes timeout script window 220, record button 222, and timeout participant window 224. Timeout script window 220 displays the timeout script which, as described above, is a summary of information relating to the scheduled procedure. In some embodiments, record button 222 may be pressed, and the timeout script may be read aloud by a nurse or other participant in the scheduled procedure, which is recorded by audio receiver 76. After the timeout script has been read, the participants listed in participant window 224 may audibly assert the accuracy of the information in the timeout script, which is also recorded by audio receiver 76. This provides additional evidence that the pre-operative verification process has been followed.

In facilities that do not or cannot provide centralized control of the functionality of components of operating room 10, alternative approaches may be employed to ensure that, prior to the commencement of a scheduled procedure, the information related to the scheduled procedure is verified by the patient and all participants in the scheduled procedure. In one alternative embodiment, system 50 is configured to sense whether components of operating room 10 have been enabled prior to completion of the pre-operative verification process and, if so, to prevent further steps in the pre-operative verification process until the enabled components of operating room 10 have been disabled. For example, light sensor 21 may be configured to send a signal to system control 60 if either or both room light fixture 20 and surgical light fixture 22 are turned on prior to completion of the pre-operative verification process, which would prevent further verification steps from occurring. Light sensor 21 may also be configured to send a signal to system control 60 when the prematurely enabled room light fixture 20 and/or surgical light fixture 22 is subsequently disabled, which would allow the pre-operative verification process to continue. Other types of sensors may also be associated with other components of operating room 10 that send signals to system control 60 when components of operating room 10 are prematurely enabled and subsequently disabled.

When the patient and all participants in the scheduled procedure have verified the information related to the scheduled procedure, system control 60 may generate a token indicating that all steps of the pre-operative verification process are completed. The token, which is unique to a particular patient and a particular scheduled procedure, may be printed out or electronically associated with the patient's record. The token may be a randomly generated, non-reproducible string of characters that cannot be guessed or generated without completion of the pre-operative verification process. The token may be stored (e.g., in server 62) with the patient's electronic record as proof that the pre-operative verification process was completed prior to commencement of the scheduled procedure. In some embodiments, remote device 70 prompts the surgeon or other participant to enter the token as a final verification. When the token is generated, stored, and/or entered, remote device 70 may alert the participants that the pre-operative verification process is complete, and components of operating room 10 may be enabled and the scheduled procedure can begin.

In summary, the present invention relates to ensuring compliance with pre-operative policies and procedures to prevent wrong site surgeries. Data related to a scheduled procedure is stored on a device. The data includes at least (1) indicia of personal identification for a patient and one or more participants in the scheduled procedure, and (2) information about the scheduled procedure. An input is received on the device that associates the patient with the data stored on the device. The patient and each of the one or more participants are then prompted to verify the indicia of personal identification and information about the scheduled procedure on the device. When the patient and one or more participants have all verified their respective indicia of personal identification and the information about the scheduled procedure, the device is operable to enable operating room functionality. By controlling operating room components in this manner, a “time-out” is mandated prior to commencement of the procedure. In addition, the order in which the information is presented to the participants may be varied between procedures, which prevents the parties involved in the procedure from starting the procedure without consciously following and conducting the pre-operative policies and procedures. Furthermore, verification of the identities of all parties involved in the procedure assures that the correct procedure is performed on the correct patient by the correct personnel.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A method for ensuring compliance with pre-operative policies and procedures to prevent wrong site surgeries, the method comprising:
storing data related to a scheduled procedure on a device, wherein the data related to the scheduled procedure includes at least (1) indicia of personal identification for a patient and one or more participants in the scheduled procedure, and (2) information about the scheduled procedure;
receiving at the device an input that associates the patient with the data stored on the device;
receiving one or more inputs from the patient at the device to verify the indicia of personal identification and information about the scheduled procedure; and
receiving one or more inputs from each of the one or more participants at the device to verify the indicia of personal identification and information about the scheduled procedure,
wherein the device is operable to enable operating room functionality when the patient and one or more participants have all verified their respective indicia of personal identification and the information about the scheduled procedure.

2. The method of claim 1, wherein receiving an input at the device that associates the patient with the data stored on the device comprises receiving a biometric input from the patient at the device.

3. The method of claim 1, wherein each of the one or more participants verifies participant-specific information about the scheduled procedure.

4. The method of claim 1, wherein each of the one or more participants verifies the information about the scheduled procedure in an order of that varies between scheduled procedures.

5. The method of claim 1, wherein after the step of receiving one or more inputs from each of the one or more participants at the device, the method further comprises:
receiving one or more inputs from at least one of the patient and the one or more participants to re-verify information about the scheduled procedure that any other of the patient and plurality of participants does not verify.

6. The method of claim 1, wherein after the step of receiving one or more inputs from each of the one or more participants at the device, the method further comprises:
receiving one or more inputs from a patient or participant who does not correctly verify the indicia of personal identification to re-verify the indicia of personal identification.

7. The method of claim 1, wherein the operating room functionality comprises any of functionality of one or more operating room lighting fixtures, functionality of one or more operating room electrical outlets, and operating room accessibility.

8. A method for controlling operating room functionality to ensure compliance with pre-operative policies and procedures designed to prevent wrong site surgeries, wherein the operating room functionality is initialized to a disabled state, the method comprising:
displaying a home screen including a selectable patient interface module and one or more selectable participant interface modules, wherein each participant interface module is associated with a participant in a scheduled procedure to be performed on a patient;
displaying a patient verification screen when the patient interface module is selected, wherein the patient verification screen includes indicia of patient personal identification and information about the scheduled procedure;
receiving one or more inputs from the patient to verify the indicia of patient personal identification and information about the scheduled procedure;
displaying a participant verification screen when a participant interface module is selected, wherein the participant verification screen displayed is specific to the participant associated with the selected participant interface module and includes indicia of participant personal identification and information about the scheduled procedure;
receiving one or more inputs from the participant associated with the selected participant interface module to verify the indicia of participant personal identification and information about the scheduled procedure; and
displaying a selectable activation module if the patient verifies the information on the patient verification screen and each participant verifies the information on the participant verification screen specific to the participant, wherein selecting the activation module enables the operating room functionality.

9. The method of claim 8, wherein displaying the participant verification screen comprises displaying a plurality of fields each relating to a different aspect of the scheduled procedure.

10. The method of claim 9, wherein an order of the plurality of fields displayed on the participant verification screen varies between scheduled procedures.

11. The method of claim 8, wherein displaying the home screen comprises:
displaying the patient interface module and the one or more selectable participant interface modules in a first color when the information on the associated verification screen has not been verified; and
displaying the patient interface module and the one or more selectable participant interface modules in a second color when the information on the associated verification screen has been verified.

12. The method of claim 8, wherein the one or more inputs received from the patient and the one or more inputs received from each participant comprises a biometric input.

13. The method of claim 8, wherein prior to displaying a selectable activation module, the method further comprises:
displaying a re-verification screen that prompts at least one of the patient and the plurality of participants to re-verify information about the scheduled procedure that any other of the patient and one or more participants does not verify.

14. The method of claim 8, wherein the operating room functionality comprises any of functionality of one or more operating room lighting fixtures, functionality of one or more operating room electrical outlets, and operating room accessibility.

15. A system for controlling operating room functionality to ensure compliance with pre-operative policies and procedures designed to prevent wrong site surgeries, the system comprising:
a server operable to store data related to a plurality of scheduled procedures, wherein, for each scheduled procedure, the data includes at least (1) indicia of personal identification for a patient and one or more participants in the scheduled procedure, and (2) information about the scheduled procedure; and
a remote device operable to receive data related to a scheduled procedure from the server and receive one or more inputs from each of the patient and one or more participants to verify the indicia of personal identification and information about the scheduled procedure, wherein the remote device facilitates enablement of operating room functionality when the patient and one or more participants have all verified their respective indicia of personal identification and the information about the scheduled procedure.

16. The system of claim 15, wherein the indicia of personal identification comprises biometric data and wherein the remote device is operable to receive a biometric input.

17. The system of claim 15, wherein the remote device is operable receive one or more inputs from each participant to verify participant-specific information about the scheduled procedure.

18. The system of claim 15, wherein the remote device is operable to prompt each participant to verify the information about the scheduled procedure in an order of that varies between scheduled procedures.

19. The system of claim 15, wherein the operating room functionality comprises any of functionality of one or more operating room lighting fixtures, functionality of one or more operating room electrical outlets, and operating room accessibility.

20. The system of claim 15, and further comprising:
a sensor associated with at least one element of the operating room functionality that sends a signal to the remote device when the at least one element is enabled.

21. The system of claim 20, wherein the remote device is operable to generate an alert if the signal is received from the sensor when fewer than all of the patient and plurality of participants have verified their respective indicia of personal identification and the information about the scheduled procedure.

22. A device for controlling operating room functionality to ensure compliance with pre-operative policies and procedures designed to prevent wrong site surgeries, the device comprising:
a controller operable to receive and store data related to a scheduled procedure, wherein the data related to the scheduled procedure includes at least (1) indicia of personal identification for a patient and one or more participants in the scheduled procedure, and (2) information about the scheduled procedure; and
at least one input element operable to receive one or more inputs from each of the patient and plurality of participants to verify the indicia of personal identification and information about the scheduled procedure, wherein the at least one input element is further operable to enable operating room functionality when the patient and plurality of participants have all verified their respective indicia of personal identification and the information about the scheduled procedure.

23. The device of claim 22, wherein the at least one input element comprises a touch screen display.

24. The device of claim 23, wherein the indicia of personal identification and information about the scheduled procedure is displayed on the touch screen display, and wherein the patient and plurality of participants are prompted to verify information on the display by pressing portions of the display.

25. The device of claim 22, wherein the at least one input element comprises a biometric input device.

26. The device of claim 25, wherein the biometric input device is operable to receive a biometric input to at least in part verify the indicia of personal identification.

27. A method for controlling operating room functionality to ensure compliance with pre-operative policies and procedures designed to prevent wrong site surgeries, wherein one or more operating room components are initialized to a disabled state, the method comprising:
storing data related to a scheduled procedure on a device, wherein the data related to the scheduled procedure includes at least (1) indicia of personal identification for a patient and one or more participants in the scheduled procedure, and (2) information about the scheduled procedure;
sensing whether the any of the one or more operating room components are switched from a disabled state to an enabled state;
receiving one or more inputs from each of the patient and the one or more participants at the device if the one or more operating room components are sensed to be in the disabled state, wherein the one or more inputs verifies the indicia of personal identification and information about the scheduled procedure; and
preventing the patient and the one or more participants from verifying the indicia of personal identification and information about the scheduled procedure if any of the one or more operating room components are sensed to be in the enabled state (a) before the patient and one or more participants have all verified their respective indicia of personal identification and the information about the scheduled procedure and (b) until the enabled one or more operating components are returned to the disabled state.

28. The method of claim 27, and further comprising:
generating a token when the patient and one or more participants have all verified their respective indicia of personal identification and the information about the scheduled procedure indicating that the one or more operating room components can be enabled.

29. The method of claim 27, wherein each of the one or more participants verifies participant-specific information about the scheduled procedure.

30. The method of claim 27, wherein each of the one or more participants verifies the information about the scheduled procedure in an order of that varies between scheduled procedures.
